(19) United States
(12) Patent Application Publication
(10) Pub. No.: US 2006/0239915 A1
(43) Pub. Date: Oct. 26, 2006

(54) Labeled macrophage scavenger receptor antagonists for imaging cardiovascular diseases

(76) Inventors: Ian Wilson, Turku (FI); Duncan Wynn, Amersham Buckinghamshire (GB)

Correspondence Address: GE HEALTHCARE, INC., IP DEPARTMENT, 101 CARNEGIE CENTER, PRINCETON, NJ, 08540-6231, US

(21) Appl. No.: 10/537,103

(22) PCT Filed: Dec. 5, 2003
(86) PCT No.: PCT/GB03/05319

§ 371 Date: Mar. 10, 2006

(30) Foreign Application Priority Data
Dec. 6, 2002 (GB) 0228490.9

(51) Int. Cl. A61B 5/055

(52) U.S. Cl. 424/9.3

(57) ABSTRACT

The present invention is in the field of diagnostic imaging. In one aspect, the invention relates to novel imaging agents comprising synthetic macrophage scavenger receptor A antagonists, said imaging agents being useful in the diagnostic imaging of cardiovascular disease. Also claimed in the present invention is a pharmaceutical composition comprising the novel imaging agents of the invention, said pharmaceutical composition being useful for the diagnostic imaging of cardiovascular disease in humans. Another aspect of the present invention is a kit useful in the preparation of the pharmaceutical composition of the invention. Furthermore, the use of the imaging agent of the invention for the diagnostic imaging of cardiovascular disease is also claimed.

### TECHNICAL FIELD OF THE INVENTION

The present invention relates to the field of in vivo diagnostic imaging. In particular the present invention relates to novel imaging agents comprising macrophage scavenger receptor antagonists, said novel imaging agents being useful in in vivo diagnostic imaging.

### BACKGROUND AND DESCRIPTION OF RELATED ART

Cardiovascular disease (CVD) is the leading cause of death in the Western world and encompasses dysfunctional conditions of the heart, arteries, veins and lungs that supply oxygen to vital life-sustaining areas of the body like the brain, the heart itself, and other vital organs. These conditions include coronary heart disease (CHD), coronary artery disease (CAD), chronic obstructive pulmonary disease (COPD), atherosclerosis, and thrombosis, and can lead to potentially life-threatening events as myocardial infarction (MI), pulmonary embolism (PE) and stroke. One factor in common to all these conditions is the involvement of macrophages.

CHD is the most prevalent of the cardiovascular diseases. In 1998 it is estimated that CHD was the cause of 7 million deaths worldwide. CAD precedes CHD, and in the majority of cases the underlying cause is atherosclerosis. Atherosclerosis is a benign disease for many decades until the atherosclerotic plaque becomes atheromatous and potentially symptom producing. The plaque can obstruct blood flow resulting in stenosis of the artery, leading to acute myocardial ischemia in the case of coronary arteries. Additionally, mature atherosclerotic plaques can rupture resulting in the release of thrombogenic lipid, and this plaque component can form a thrombosis that completely blocks the artery. Angina is a common manifestation of CHD and is often the forerunner to more serious complications such as acute coronary syndromes including unstable angina, myocardial infarction and sudden cardiac death. Plaque rupture precedes the majority of clinical events and the vulnerability of plaques is the most important predictor of clinical outcome.

Macrophage scavenger receptors (MSRs) are expressed on resident macrophages in tissues such as lung, liver, spleen, and recognise modified forms of low-density lipoprotein (LDL). They are not expressed on circulating cells. Class A MSR (MSRA) is known to have a role in the development of atherosclerotic plaques, MSRA I and MSRA II being responsible for the uptake of oxidised LDL and acetylated LDL into macrophages. MSRA expression is an indicator of the lipid burden of macrophages, and therefore may indicate instability of an atherosclerotic plaque.

A series of MSRA antagonists have been reported as being useful in the treatment of CVD. These include salicylanilide derivatives (WO 99/07382), isophthalic acid derivatives (WO 00/06147), phenylenediamines (WO 00/03704) and sulfonamidobenzanilide derivatives (WO 00/78145 and WO 01/98264). The cited documents disclose pharmaceutical compositions comprising these compounds for the treatment of CVD in humans. In addition to being useful in the treatment of CVD, the cited documents also disclose that these compounds may be used in methods for antagonising the MSRA in animals as well as methods for inhibiting lipid accumulation within macrophage-derived foam cells.

WO 02/067761 discloses detectably labelled MSRA antagonists as being useful in the diagnosis and monitoring of CVD. The MSRA antagonists of WO 02/067761 are salicylanilide derivatives, isophthalic acid derivatives and phenylenediamine derivatives. MSRA antagonists that are sulphonamidobenzamide compounds are not disclosed. The IC₅₀ values for the compounds of WO 02/067761 are disclosed as <100 mM in binding/uptake assays. No specific examples of particular compounds tested are given in that document. The compounds of the present invention have been shown to display superior binding characteristics in comparison to those reported in WO 02/067761.

### SUMMARY OF THE PRESENT INVENTION

Novel imaging agents comprising synthetic MSRA antagonists have now been identified that possess superior properties over the prior art compounds for diagnosis and monitoring of CVD as well as neurological conditions in which microglia are involved.

An MSRA antagonist is attached to an imaging moiety, said imaging moiety being suitable for the in vivo detection of the MSRA antagonist using known diagnostic imaging modalities. Suitable synthetic MSRA antagonists of the present invention are sulphonamidobenzamide compounds. The imaging agents of the invention display superior properties for imaging compared with the prior art compounds.

Also disclosed in the present invention is a pharmaceutical composition comprising the novel imaging agent of the present invention and kits for the preparation of said pharmaceutical composition. Furthermore, the present invention discloses a method of imaging CVD using the novel imaging agent of the invention.

### DETAILED DESCRIPTION OF THE INVENTION

The compounds of the invention are useful for diagnostic imaging of CVD. “CVD” as defined in the present invention includes such disease states as atherosclerosis, CAD, thrombosis, transient ischaemia and renal disease. The compounds of the invention are also useful for diagnostic imaging of neurological diseases where monocyte-derived nervous system cells called microglia are implicated such as Alzheimer's disease, Parkinson's disease, multiple sclerosis and encephalitis.

A first aspect of the invention is an imaging agent which comprises a synthetic MSRA antagonist labelled with an imaging moiety, wherein the synthetic MSRA antagonist is a sulphonamidobenzamide compound, and wherein the imaging moiety can be detected externally in a non-invasive manner following administration of said labelled synthetic MSRA antagonist to the mammalian body in vivo.

Suitable sulphonamidobenzamide compounds of the invention are of Formula (I): 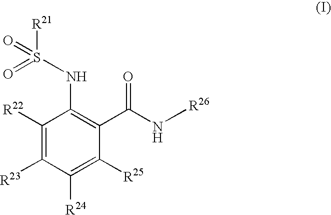
wherein R²¹ to R²⁶ are independently selected from hydrogen, C₁₋₆ alkyl, C₆₋₁₄ aryl, carboxy, amino, hydroxy, or methoxy and wherein one or more of R²² to R²⁵ may alternatively be a halogen.

A preferred sulphonamidobenzamide compound of the invention is of Formula (II): 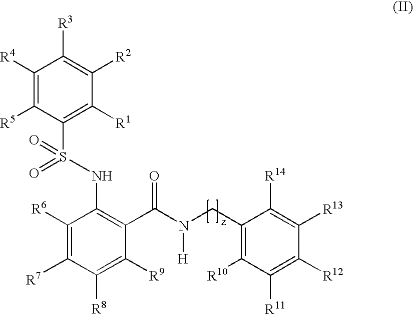
wherein;
z is 0, 1 or 2;
R¹-R¹⁴ are independently R groups, where R is;
   hydrogen, hydroxy, carboxy, C₁₋₆ alkyl, nitro, cyano, amino, halogen, C₆₋₁₄ aryl, alkenyl, alkynyl, acyl, aroyl, carboalkoxy, carbamoyl, carbamyl, alkysulphinyl, arylsulphinyl, arylalkylsulphinyl, alkylsulphonyl, arylsulphonyl, arylalkylsulphonyl, sulphamyl, arylsulphonamido or alkylsulphonamido.

A preferred imaging agent of the invention is of Formula (II) wherein each R¹ to R¹⁴ is chosen from an imaging moiety, hydrogen, C₁₋₆ alkyl, hydroxy, carboxy, amino or halogen.

A most preferred imaging agent of the invention is of Formula (II) wherein one of R², R³, R⁷, R⁸ and R¹² is an imaging moiety, and the remaining R², R³, R⁷, R⁸ and R¹² groups are independently selected from hydrogen, C₁₋₆ alkyl, carboxy, or a halogen selected from chlorine, bromine, fluorine or iodine.

An especially preferred imaging agent of the invention is of Formula (II) wherein R³, R⁸ and R¹² are all halogens with at least one being an imaging moiety.

The sulphonamidobenzamide compounds of the invention can be prepared as described in Scheme 1 of WO 00/78145. A synthesis of a compound of Formula (II) where z=0 is illustrated in FIG. 1. R¹ to R¹⁴ are as defined for Formula (II) above. Similar syntheses may be used for the preparation of compounds of Formula (II) wherein z=1 and z=2.

“Alkyl” used either alone or as part of another group is defined herein as any straight, branched or cyclic, saturated or unsaturated CₙH₂ₙ₊₁ group, wherein unless otherwise specified n is an integer between 1 and 6. The term alkyl in the present invention is also taken to include substituted alkyls, e.g. hydroxyalkyls, haloalkyls, aminoalkyls, carboxyalkyls and alkoxyalkyls.

“Aryl” used either alone or as part of another group is defined herein as any C₆₋₁₄ molecular fragment or group which is derived from a monocyclic or polycyclic aromatic hydrocarbon. Suitable aryl groups of the invention include, but are not limited to, haloaryl, alkylaryl, arylcarbamyl, phenylazo, arylamino, arylthio, toluene, benzoic acid, phenol, arylsulfinyl, arylsulfonyl, arylsulfonamido, benzothiophene, naphthalene, quinoline, isoquinoline, pyridine, pyrimidine, and pyrazine.

The term “halogen” means a group selected from fluorine, chlorine, bromine, and iodine or isotopes thereof.

The “imaging moiety” is preferably chosen from:
(i) a radioactive metal ion;
(ii) a paramagnetic metal ion;
(iii) a gamma-emitting radioactive halogen;
(iv) a positron-emitting radioactive non-metal;
(v) a hyperpolarised NMR-active nucleus;
(vi) a reporter suitable for in vivo optical imaging;
(vii) a β-emitter suitable for intravascular detection.

When the imaging moiety is a radioactive metal ion, i.e. a radiometal, suitable radiometals can be either positron emitters such as ⁶⁴Cu, ⁴⁸V, ⁵²Fe, ⁵⁵Co, ^{94m}Tc or ⁶⁸Ga; γ-emitters such as ^{99m}Tc, ¹¹¹In, ^{113m}In, or ⁶⁷Ga. Preferred radiometals are ^{99m}Tc, ⁶⁴Cu, ⁶⁸Ga and ¹¹¹In. Most preferred radiometals are γ-emitters, especially ^{99m}Tc.

When the imaging moiety is a paramagnetic metal ion, suitable such metal ions include: Gd(III), Mn(II), Cu(II), Cr(II), Fe(III), Co(II), Er(II), Ni(II), Eu(III) or Dy(III). Preferred paramagnetic metal ions are Gd(III), Mn(II) and Fe(III), with Gd(III) being especially preferred.

When the imaging moiety is a gamma-emitting radioactive halogen, the radiohalogen is suitably chosen from ¹²³I, ¹²⁵I, ¹³¹I or ⁷⁷Br. A preferred gamma-emitting radioactive halogen is ¹²³I.

When the imaging moiety is a positron-emitting radioactive non-metal, suitable such positron emitters include: ¹¹C, ¹³N, ¹⁵O, ¹⁷F, ¹⁸F, ⁷⁵Br, ⁷⁶Br or ¹²⁴I. Preferred positron-emitting radioactive non-metals are ¹¹C, ¹³N and ¹⁸F, especially ¹¹C and ¹⁸F, most especially ¹⁸F.

When the imaging moiety is a hyperpolarised NMR-active nucleus, such NMR-active nuclei have a non-zero nuclear spin, and include ¹³C, ¹⁵N, ¹⁹F, ²⁹Si and ³¹P. Of these, ¹³C is preferred. By the term “hyperpolarised” is meant enhancement of the degree of polarisation of the NMR-active nucleus over its' equilibrium polarisation. The natural abundance of ¹³C (relative to ¹²C) is about 1%, and suitable ¹³C-labelled compounds are suitably enriched to an abundance of at least 5%, preferably at least 50%, most preferably at least 90% before being hyperpolarised. At least one carbon atom of the sulphonamidobenzamide compound of the present invention is suitably enriched with ¹³C, which is subsequently hyperpolarised.

When the imaging moiety is a reporter suitable for in vivo optical imaging, the reporter is any moiety capable of detection either directly or indirectly in an optical imaging procedure. The reporter might be a light scatterer (e.g. a coloured or uncoloured particle), a light absorber or a light emitter. More preferably the reporter is a dye such as a chromophore or a fluorescent compound. The dye can be any dye that interacts with light in the electromagnetic spectrum with wavelengths from the ultraviolet light to the near infrared. Most preferably the reporter has fluorescent properties.

Preferred organic chromophoric and fluorophoric reporters include groups having an extensive delocalized electron system, e.g. cyanines, merocyanines, indocyanines, phthalocyanines, naphthalocyanines, triphenylmethines, porphyrins, pyrilium dyes, thiapyriliup dyes, squarylium dyes, croconium dyes, azulenium dyes, indoanilines, benzophenoxazinium dyes, benzothiaphenothiazinium dyes, anthraquinones, napthoquinones, indathrenes, phthaloylacridones, trisphenoquinones, azo dyes, intramolecular and intermolecular charge-transfer dyes and dye complexes, tropones, tetrazines, bis(dithiolene) complexes, bis(benzene-dithiolate) complexes, iodoaniline dyes, bis(S,O-dithiolene) complexes. Fluorescent proteins, such as green fluorescent protein (GFP) and modifications of GFP that have different absorption/emission properties are also useful. Complexes of certain rare earth metals (e.g., europium, samarium, terbium or dysprosium) are used in certain contexts, as are fluorescent nanocrystals (quantum dots).

Particular examples of chromophores which may be used include: fluorescein, sulforhodamine 101 (Texas Red), rhodamine B, rhodamine 6G, rhodamine 19, indocyanine green, Cy2, Cy3, Cy3.5, Cy5, Cy5.5, Cy7, Marina Blue, Pacific Blue, Oregon Green 88, Oregon Green 514, tetramethylrhodamine, and Alexa Fluor 350, Alexa Fluor 430, Alexa Fluor 532, Alexa Fluor 546, Alexa Fluor 555, Alexa Fluor 568, Alexa Fluor 594, Alexa Fluor 633, Alexa Fluor 647, Alexa Fluor 660, Alexa Fluor 680, Alexa Fluor 700, and Alexa Fluor 750.

Particularly preferred are dyes which have absorption maxima in the visible or near infrared region, between 400 nm and 3 μm, particularly between 600 and 1300 nm.

Optical imaging modalities and measurement techniques include, but not limited to: luminescence imaging; endoscopy; fluorescence endoscopy; optical coherence tomography; transmittance imaging; time resolved transmittance imaging; confocal imaging; nonlinear microscopy; photoacoustic imaging; acousto-optical imaging; spectroscopy; reflectance spectroscopy; interferometry; coherence interferometry; diffuse optical tomography and fluorescence mediated diffuse optical tomography (continuous wave, time domain and frequency domain systems), and measurement of light scattering, absorption, polarisation, luminescence, fluorescence lifetime, quantum yield, and quenching.

When the imaging moiety is a β-emitter suitable for intravascular detection, suitable such β-emitters include the radiometals ⁶⁷Cu, ⁸⁹Sr, ⁹⁰Y, ¹⁵³Sm, ¹⁸⁶Re, ¹⁸⁸Re or ¹⁹²Ir, and the non-metals ³²P, ³³P, ³⁸S, ³⁸Cl, ³⁹Cl, ⁵²Br and ⁸³Br.

Whichever imaging moiety is selected from the above, it is preferably reacted with a precursor of said imaging agent. An imaging agent precursor constitutes a second aspect of the present invention. Reaction of such a precursor with a suitable chemical form of the imaging moiety results in the production of said imaging agent. A “precursor” as defined in the present invention is a MSRA antagonist compound to which an imaging moiety may be readily attached, preferably in a one-step process. One example of a suitable precursor of the invention is a MSRA antagonist conjugated to a metal chelating agent, suitable for the attachment of an imaging moiety which is a metal ion. Another example of a suitable precursor of the invention is a MSRA antagonist that includes a group such as (a) a non-radioactive halogen atom, (b) an activated aryl ring, (c) an organometallic precursor compound, or (d) an organic precursor such as triazene. Such a precursor is suitable for the incorporation of an imaging moiety which is a radioactive halogen. These precursor compounds and the resultant imaging agents are described more fully in the following sections.

When the imaging moiety comprises a metal ion, the metal ion is suitably attached to the MSRA antagonist as part of a conjugated metal complex of Formula (III):
[{MSRA antagonist}-(L)ₓ]_{y}-[metal complex] (III)
wherein:
-(L)ₓ- is a linker group wherein each L is independently —CZ₂-, —CZ=CZ-, —C≡C—, —CZ₂CO₂—, —CO₂CZ₂-, —NZCO—, —CONZ-, —NZ(C═O)NZ-, —NZ(C═S)NZ-, —SO₂NZ-, —NZSO₂—, —CZ₂OCZ₂-, —CZ₂SCZ₂-, —CZ₂NZCZ₂-, a C₄₋₈ cycloheteroalkylene group, a C₄₋₈ cycloalkylene group, a C₃₋₁₂ arylene group, a C₃₋₁₂ heteroarylene group, an amino acid or a monodisperse polyethyleneglycol (PEG) building block;
Z is independently chosen from H, C₁₋₄ alkyl, C₂₋₄ alkenyl, C₂₋₄ alkynyl, C₁₋₄ alkoxyalkyl or C₁₋₄ hydroxyalkyl;
x is an integer of value 0 to 10; and
y is 1, 2 or 3.

By the term “metal complex” is meant a coordination complex of the metal ion with one or more ligands. It is strongly preferred that the metal complex is “resistant to transchelation”, i.e. does not readily undergo ligand exchange with other potentially competing ligands for the metal coordination sites. Potentially competing ligands include the synthetic MSRA antagonist itself plus other excipients in the preparation in vitro (e.g. radioprotectants or antimicrobial preservatives used in the preparation), or endogenous compounds in vivo (e.g. glutathione, transferrin or plasma proteins). The “linker group” (L)ₓ is as defined below for Formula (IIIa).

The metal complexes of Formula (III) are conveniently prepared from precursors which are ligand conjugates of Formula (IIIa):
[{MSRA antagonist}-(L)ₓ]_{y}-[ligand] (IIIa)
wherein (L)ₓ, x and y are as defined for Formula (III) above.

In Formulae (III) and (IIIa), y is preferably 1 or 2, and is most preferably 1.

Suitable ligands for use in the present invention, which form metal complexes resistant to transchelation, include chelating agents which have 2-6, preferably 2-4, metal donor atoms arranged such that 5- or 6-membered chelate rings result (by having a non-coordinating backbone of either carbon atoms or non-coordinating heteroatoms linking the metal donor atoms). Examples of donor atom types which bind well to metals as part of chelating agents are: amines, thiols, amides, oximes and phosphines. Phosphines form such strong metal complexes that even monodentate or bidentate phosphines form suitable metal complexes. The linear geometry of isonitriles and diazenides is such that they do not lend themselves readily to incorporation into chelating agents, and are hence typically used as monodentate ligands. Examples of suitable isonitriles include simple alkyl isonitriles such as tert-butylisonitrile, and ether-substituted isonitriles such as MIBI (i.e. 1-isocyano-2-methoxy-2-methylpropane). Examples of suitable phosphines include Tetrofosmin, and monodentate phosphines such as tris(3-methoxypropyl)phosphine. Examples of suitable diazenides include the HYNIC series of ligands, i.e. hydrazine-substituted pyridines or nicotinamides.

Examples of suitable chelating agents for technetium which form metal complexes resistant to transchelation include, but are not limited to:
(i) diaminedioximes of Formula (IV): 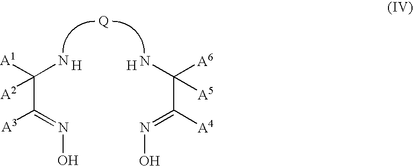
where A¹-A⁶ are each independently an A group;
each A is H or C₁₋₁₀ alkyl, C₃₋₁₀ alkylaryl, C₂₋₁₀ alkoxyalkyl, C₁₋₁₀ hydroxyalkyl, C₁₋₁₀ fluoroalkyl, C₂₋₁₀ carboxyalkyl or C₁₋₁₀ aminoalkyl, or two or more A groups together with the atoms to which they are attached form a carbocyclic, heterocyclic, saturated or unsaturated ring, and wherein one or more of the A groups is conjugated to the MSRA antagonist;
and Q is a bridging group of Formula -(J)ₘ-;
where m is 3, 4 or 5 and each J is independently —O—, —NA- or —C(A)₂- provided that -(J)ₘ- contains a maximum of one J group which is —O— or —NA-.

Preferred Q groups are as follows:

Q=—(CH₂)(CHA)(CH₂)— i.e. propyleneamine oxime or PnAO derivatives;

Q=—(CH₂)₂(CHA)(CH₂)₂— i.e. pentyleneamine oxime or PentAO derivatives;

Q=—(CH₂)₂NA(CH₂)₂—.

A¹ to A⁶ are preferably chosen from: C₁₋₃ alkyl, alkylaryl alkoxyalkyl, hydroxyalkyl, fluoroalkyl, carboxyalkyl or aminoalkyl. Most preferably, each A¹ to A⁶ group is CH₃.

The synthetic MSRA antagonist is preferably conjugated at either A¹ or A⁶, or an A group of the Q moiety. Most preferably, the MSRA antagonist is conjugated to an A group of the Q moiety. When the MSRA antagonist is conjugated to an A group of the Q moiety, the A group is preferably at the bridgehead position. In that case, Q is preferably —(CH₂)(CHA)(CH₂)—, —(CH₂)₂(CHA)(CH₂)₂— or —(CH₂)₂(NA)(CH₂)₂—, most preferably —(CH₂)₂(CHA)(CH₂)₂—.

An especially preferred bifunctional diaminedioxime chelator is of Formula (V):

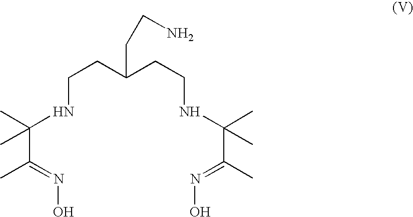

This chelator will also be referred to as “chelating agent 1”. The synthetic MSRA antagonist is conjugated to chelating agent 1 via the bridgehead NH₂ group.

(ii) N₃S₂ ligands having a thioltriamide donor set such as MAG₃ and related ligands; or having a diamidepyridinethiol donor set such as PICA;

(iii) N₂S₂ ligands having a diaminedithiol donor set such as BAT or ECD (i.e. ethylcysteinate dimer), or an amideaminedithiol donor set such as MAMA;

(iv) N₄ ligands which ore open chain or macrocyclic ligands having a tetramine, amidetriamine or diamidediamine donor set, such as cyclam, monoxocyclam or dioxocyclam; and,

(v) N₂O₂ ligands having a diaminediphenol donor set.

The above described ligands are particularly suitable for complexing technetium, e.g. ^{94m}Tc or ^{99m}Tc, and are described more fully by Jurisson et al [Chem. Rev., 99, 2205-2218 (1999)]. The ligands are also useful for other metals, such as copper (⁶⁴Cu or ⁶⁷Cu), vanadium (e.g. ⁴⁸V), iron (e.g. ⁵²Fe), or cobalt (e.g. ⁵⁵CO). Other suitable ligands are described in Sandoz WO 91/01144, which includes ligands which are particularly suitable for indium, yttrium and gadolinium, especially macrocyclic aminocarboxylate and aminophosphonic acid ligands. Ligands which form non-ionic (i.e. neutral) metal complexes of gadolinium are known and are described in U.S. Pat. No. 4,885,363. When the radiometal ion is technetium, the ligand is preferably a chelating agent which is tetradentate. Preferred chelating agents for technetium are the diaminedioximes, or those having an N₂S₂ or N₃S donor set as described above. Especially preferred chelating agents for technetium are the diaminedioximes.

It is envisaged that the role of the linker group -(L)ₓ- in Formula (III) and (IIIa) is to distance the relatively bulky metal complex which results upon metal co-ordination, from the active site of the MSRA antagonist, so that binding of the antagonist to MSRA is not impaired. This can be achieved by a combination of flexibility (e.g. simple alkyl chains), so that the bulky group has the freedom to position itself away from the active site and/or rigidity such as a cycloalkyl or aryl spacer which orientates the metal complex away from the active site.

The nature of the linker group can also be used to modify the biodistribution of the resulting metal complex of the conjugate. Thus, e.g. the introduction of ether groups in the linker will help to minimise plasma protein binding. Preferred linker groups have a backbone chain of linked atoms which make up the (L)ₓ moiety containing 2 to 10 atoms, most preferably 2 to 5 atoms, with 2 or 3 atoms being especially preferred. A minimum linker group backbone chain of 2 atoms confers the advantage that the ligand is well-separated from the MSRA antagonist so that any interaction is minimised.

Non-peptide linker groups such as alkylene groups or arylene groups have the advantage that there are no significant hydrogen bonding interactions with the conjugated MSRA antagonist, so that the linker does not wrap round onto the MSRA antagonist. Preferred alkylene spacer groups are (CH₂)_{q} where q is 2 to 5. Preferred arylene spacers are of Formula (VI): 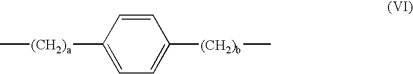
where: a and b are independently 0, 1 or 2.

It is strongly preferred that the metal complex is bound in such a way that the linkage does not undergo facile metabolism in blood, since that would result in the metal complex being cleaved off before the imaging agent reaches the desired in vivo target site. The metal complexes are preferably covalently bound via linkages which are not readily metabolised.

When the imaging moiety is a radioactive halogen, it is preferably a radioactive isotope of iodine. The radioiodine atom is preferably attached via a direct covalent bond to an aromatic ring such as a benzene ring, or a vinyl group since it is known that iodine atoms bound to saturated aliphatic systems are prone to in vivo metabolism and hence loss of the imaging moiety.

When the imaging moiety is radioactive halogen, such as iodine, suitable precursors of the imaging agent include: a non-radioactive halogen atom such as an aryl iodide or aryl bromide (to permit radioiodine exchange); an activated aryl ring (e.g. a phenol group); or an organometallic precursor compound (e.g. trialkyltin or trialkylsilyl), an organic precursor such as triazenes or other such moiety known to those skilled in the art. Methods of introducing radioactive halogens (including ¹²³I and ¹⁸F) are described by Bolton [J. Lab. Comp. Radiopharm., 45, 485-528 (2002)].

Examples of suitable aryl groups to which radioactive halogens, especially iodine can be attached are given below:

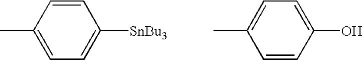

Both contain substituents which permit facile radioiodine substitution onto the aromatic ring. Alternative substituents containing radioactive iodine can be synthesised by direct iodination, e.g. via radiohalogen exchange:

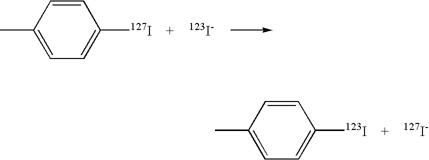

When the imaging moiety comprises a radioactive isotope of fluorine (eg. ¹⁸F), the radioiodine atom may be carried out via direct labelling using the reaction of ¹⁸F-fluoride with a suitable precursor having a good leaving group, such as an alkyl bromide, alkyl mesylate or alkyl tosylate. ¹⁸F can also be introduced by N-alkylation of amine precursors with alkylating agents such as ¹⁸F(CH₂)₃OMs (where Ms is mesylate) to give N—(CH₂)₃¹⁸F, or O-alkylation of hydroxyl groups with ¹⁸F(CH₂)₃OMs or ¹⁸F(CH₂)₃Br. For aryl systems, ¹⁸F-fluoride displacement of nitrogen from an aryl diazonium salt is a possible route to aryl-18F derivatives. See Bolton, J. Lab. Comp. Radiopharm., 45, 485-528 (2002) for a description of routes to ¹⁸F-labelled derivatives.

In a third aspect of the invention a pharmaceutical composition comprising the imaging agent of the invention together with a biocompatible carrier, in a form suitable for mammalian administration, is disclosed.

A “pharmaceutical composition” is defined in the present invention as a formulation comprising the imaging agent of the invention or a salt thereof in a form suitable for administration to humans. The pharmaceutical composition of the invention is preferably administered parenterally, i.e. by injection, and most preferably as an aqueous solution. Such a formulation may optionally contain further ingredients such as buffers; pharmaceutically acceptable solubilisers (e.g. cyclodextrins or surfactants such as Pluronic, Tween or phospholipids); pharmaceutically acceptable stabilisers or antioxidants (such as ascorbic acid, gentisic acid or para-aminobenzoic acid).

In a fourth aspect of the invention, a kit for the preparation of the pharmaceutical composition of the invention is disclosed which comprises a precursor of the imaging agent of the invention. Such kits are designed to give sterile products suitable for human administration, e.g. via direct injection into the bloodstream, and comprise a precursor of said imaging agent.

Preferably, the kit is for the preparation of a pharmaceutical composition which comprises an imaging agent wherein the imaging moiety is selected from a radioactive metal ion, a paramagnetic metal ion, or a radiohalogen. The precursor in each case is as described earlier in the description, e.g. Formula (IIIa) for metal ions.

Where the radiometal is ^{99m}Tc, the kit is preferably lyophilised and is designed to be reconstituted with sterile ^{99m}Tc-pertechnetate (TcO₄⁻) from a ^{99m}Tc radioisotope generator to give a solution suitable for human administration without further manipulation. Suitable kits comprise a container (e.g. a septum-sealed vial) containing the MSRA antagonist-chelating agent conjugate in either free base or acid salt form, together with a pharmaceutically acceptable reducing agent such as sodium dithionite, sodium bisulphite, ascorbic acid, formamidine sulphinic acid, stannous ion, Fe(II) or Cu(I). The pharmaceutically acceptable reducing agent is preferably a stannous salt such as stannous chloride or stannous tartrate. Alternatively, the kit may optionally contain a metal complex, which upon addition of the radiometal, undergoes transmetallation (i.e. metal exchange) giving the desired product.

Kits for the preparation of the imaging agents of the invention may optionally further comprise additional components such as a transchelator, radioprotectant, antimicrobial preservative, pH-adjusting agent or filler. The “transchelator” is a compound which reacts rapidly to form a weak complex with technetium, then is displaced by the diaminedioxime. This minimises the risk of formation of reduced hydrolysed technetium (RHT) due to rapid reduction of pertechnetate competing with technetium complexation.

Suitable such transchelators are salts of a weak organic acid, i.e. an organic acid having a pKa in the range 3 to 7, with a biocompatible cation. Suitable such weak organic acids are acetic acid, citric acid, tartaric acid, gluconic acid, glucoheptonic acid, benzoic acid, phenols or phosphonic acids. Hence, suitable salts are acetates, citrates, tartrates, gluconates, glucoheptonates, benzoates, phenolates or phosphonates. Preferred such salts are tartrates, gluconates, glucoheptonates, benzoates, or phosphonates, most preferably phosphonates, most especially diphosphonates. A preferred such transchelator is a salt of MDP, i.e. methylenediphosphonic acid, with a biocompatible cation.

By the term “radioprotectant” is meant a compound which inhibits degradation reactions, such as redox processes, by trapping highly-reactive free radicals, such as oxygen-containing free radicals arising from the radiolysis of water. The radioprotectants of the present invention are suitably chosen from: ascorbic acid, para-aminobenzoic acid (i.e. 4-aminobenzoic acid), gentisic acid (i.e. 2,5-dihydroxybenzoic acid) and salts thereof with a biocompatible cation as described above.

By the term “antimicrobial preservative” is meant an agent which inhibits the growth of potentially harmful micro-organisms such as bacteria, yeasts or moulds. The antimicrobial preservative may also exhibit some bactericidal properties, depending on the dose. The main role of the antimicrobial preservative(s) of the present invention is to inhibit the growth of any such micro-organism in the pharmaceutical composition post-reconstitution, i.e. in the imaging agent itself. The antimicrobial preservative may, however, also optionally be used to inhibit the growth of potentially harmful micro-organisms in one or more components of the kit of the present invention prior to reconstitution. Suitable antimicrobial preservative(s) include: the parabens, i.e. methyl, ethyl, propyl or butyl paraben or mixtures thereof; benzyl alcohol; phenol; cresol; cetrimide and thiomersal. Preferred antimicrobial preservative(s) are the parabens.

The term “pH-adjusting agent” means a compound or mixture of compounds useful to ensure that the pH of the reconstituted kit is within acceptable limits (approximately pH 4.0 to 10.5) for human or mammalian administration. Suitable such pH-adjusting agents include pharmaceutically acceptable buffers, such as tricine, phosphate or TRIS [i.e. tris(hydroxymethyl)aminomethane], and pharmaceutically acceptable bases such as sodium carbonate, sodium bicarbonate or mixtures thereof. When the MSRA antagonist-chelating agent conjugate is employed in acid salt form, the pH adjusting agent may optionally be provided in a separate vial or container, so that the user of the kit can adjust the pH as part of a multi-step procedure.

By the term “filler” is meant a pharmaceutically acceptable bulking agent which may facilitate material handling during production and lyophilisation. Suitable fillers include inorganic salts such as sodium chloride, and water soluble sugars or sugar alcohols such as sucrose, maltose, mannitol or trehalose.

A fifth aspect of the present invention is the use of the pharmaceutical composition of the invention for the diagnostic imaging of CVD. Preferably, the pharmaceutical composition of the invention may be used in the diagnostic imaging of atherosclerotic plaques, coronary artery disease, thrombosis, transient ischaemia or renal disease. Most preferably, the pharmaceutical composition of the invention may be used in the diagnostic imaging of atherosclerotic plaques. An especially preferred use of the pharmaceutical composition of the invention is for the diagnostic imaging of unstable atherosclerotic plaques.

A further use of the pharmaceutical composition of the invention is in the diagnostic imaging of neurological diseases in which microglial cells are involved, such as Alzheimer's disease, multiple sclerosis, Parkinson's disease and encephalitis.

### BRIEF DESCRIPTION OF THE FIGURES

FIG. 1 illustrates the general synthetic route used to obtain the sulphonamidobenzamide compounds of the invention.

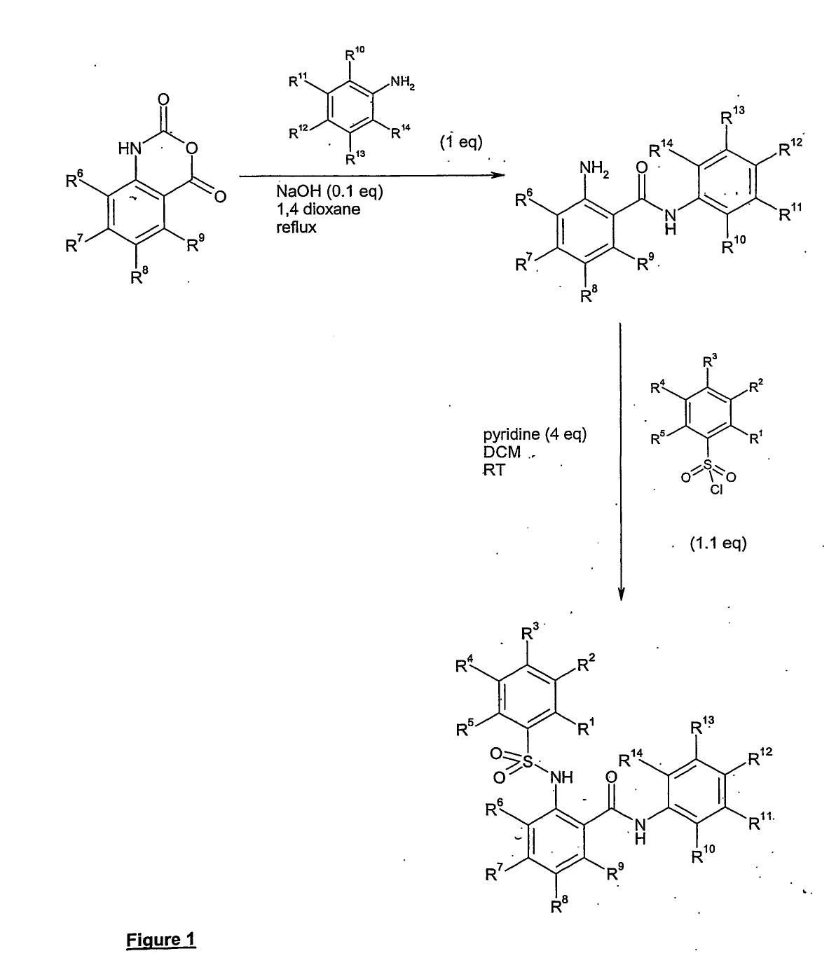

FIG. 2 illustrates the synthetic route used to obtain precursor 1 and the ^{99m}Tc-labelled imaging agent 1.

FIG. 3 illustrates the chemical structures of precursor 2 and precursor 3, both of which are suitable for labelling with ^{99m}Tc.

FIG. 4 illustrates the synthetic route used to obtain precursor 4 and the radioiodinated imaging agent 2.

FIG. 5 illustrates the synthetic route used to obtain precursor 5 and the ¹⁸F-labelled imaging agent 3.

FIG. 6 shows competition of various agents for binding of ¹²⁵I-imaging agent 2 to THP-1 and CHO-SRA cells. The various agents are (A) non-radioactive imaging agent 2 (B) AcLDL and (C) OxLDL.

FIG. 7 shows the binding of ¹²⁵I-imaging agent 2 to THP-1 Cells using a spiked assay format.

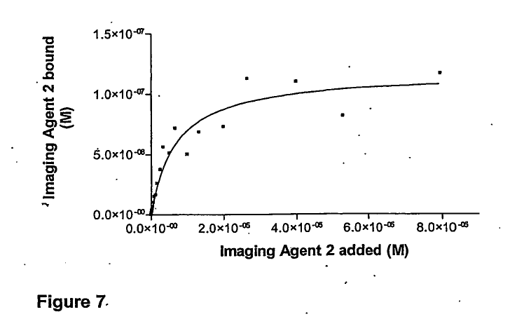

FIG. 8 shows the biodistribution of ¹²⁵I-imaging agent 2 in rats over a period of 4 hours post injection (p.i.).

### BRIEF DESCRIPTION OF THE EXAMPLES

Various embodiments of the invention are described in the following non-limiting examples.

Example 1 relates to the synthesis of chelating agent 1, which is then used in the preparation of precursor 1 in Example 2. Precursor 1 is a compound suitable for the attachment of a metal ion, preferably ^{99m}Tc, the attachment of which is described in Example 5. Examples 3 and 4 describe the synthesis of precursors 2 and 3, both suitable for labelling with ^{99m}Tc.

Example 6 describes the synthesis of precursor 4, a compound which is suitable for straightforward substitution with radiohalogen. The process of radioiodinating precursor 4 to form imaging agent 2 is described in Example 7.

Example 8 describes the synthesis of precursor 5, which is suitable for radiofluorination. Example 9 describes a method of preparing the ¹⁸F compound from precursor 5.

Examples 10 and 11 describe how the non-radioactive reference versions of imaging agents 2 and 3 were prepared. These non-radioactive versions were used in the scavenger receptor binding assay because their binding characteristics would be identical to the radioiodinated imaging agents 2 and 3.

Example 12 outlines the method used to assess the binding characteristics of compounds of the invention. IC50 values of <40 μM for were found in this binding assay for the non radioactive versions of imaging agents 2 and 3.

Example 13 and 14 describe the cell binding assay used to evaluate binding of ¹²⁵I imaging agent 2 to cells expressing MSRA. In example 13, ¹²⁵I imaging agent 2 was found to bind to MSRA present on THP-1 and CHO-SRA cells. Saturation binding of ¹²⁵I-imaging agent 2 to THP-1 cells fit to and was analysed by a one site binding curve, a K_{d} of 6.74 μM was calculated. Therefore in contrast to the competition data, ¹²⁵I imaging agent 2 appears to bind with μM affinity. In example 14, binding of ¹²⁵I imaging agent 2 was competed by cold ¹²⁷I imaging agent 2 and AcLDL demonstrating specific binding of the agent to MSRA.

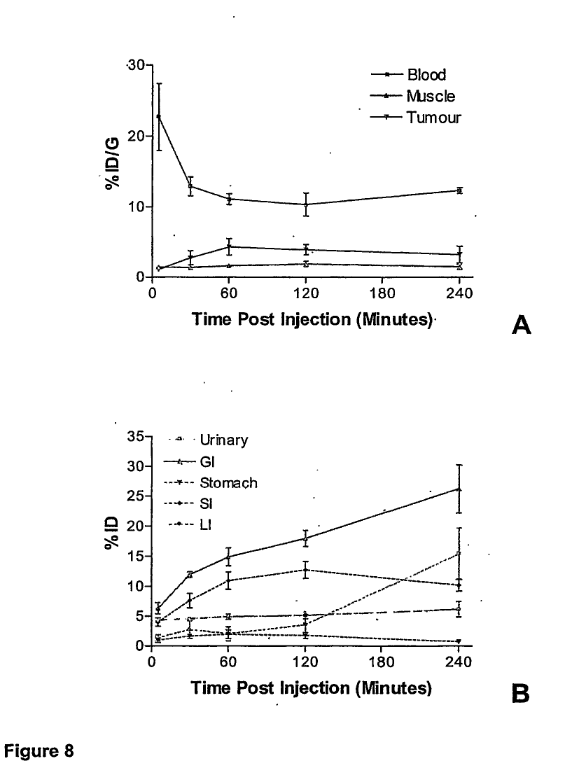

Example 15 describes the in vivo characteristics of ¹²⁵I imaging agent 2 in a mouse tumour model. Results showed minimal de-iodination in vivo, approximately 20% up to 4 hours p.i. Blood retention was high throughout with a slow rate of excretion, with higher GI excretion (FIGS. 8A and 8B). Uptake of ¹²⁵I imaging agent 2 into tumour tissue was initially low but increased to peak at 60 minutes p.i. and remained constant thereon. Increased ¹²⁵I imaging agent 2 at tumour site was most likely specific as blood retention decreased from 5-60 minutes p.i. Good tumour to muscle ratios were obtained, with optimal ratio seen at 60 minutes p.i. (FIG. 8A).

### EXAMPLE 1

### Synthesis of Chelating Agent 1

### Step 1(a): 3(methoxycarbonylmethylene)glutaric acid dimethylester

Carbomethoxymethylenetriphenylphosphorane (167 g, 0.5 mol) in toluene (600 ml) was treated with dimethyl 3-oxoglutarate (87 g, 0.5 mol) and the reaction heated to 100° C. on an oil bath at 120° C. under an atmosphere of nitrogen for 36 h. The reaction was then concentrated in vacuo and the oily residue triturated with 40/60 petrol ether/diethylether 1:1, 600 ml. Triphenylphosphine oxide precipitated out and the supernatant liquid was decanted/filtered off. The residue on evaporation in vacuo was Kugelrohr distilled under high vacuum Bpt (oven temperature 180-200° C. at 0.2 torr) to give 3(methoxycarbonylmethylene)glutaric acid dimethylester in 89.08 g, 267 mM, 53%.

NMR ¹H(CDCl₃): δ 3.31 (2H, s, CH₂), 3.7(9H, s, 3×OCH₃), 3.87 (2H, s, CH₂), 5.79 (1H, s, ═CH,) ppm.

NMR ¹³C(CDCl₃), δ 36.56, CH₃, 48.7, 2×CH₃, 52.09 and 52.5 (2×CH₂); 122.3 and 146.16 C═CH; 165.9, 170.0 and 170.5 3×COO ppm.

### Step 1 (b): Hydrogenation of 3-(methoxycarbonylmethylene)glutaric acid dimethylester

3(methoxycarbonylmethylene)glutaric acid dimethylester (89 g, 267 mmol) in methanol (200 ml) was shaken with (10% palladium on charcoal: 50% water) (9 g) under an atmosphere of hydrogen gas (50 psi) for (30 h). The solution was filtered through kieselguhr and concentrated in vacuo to give 3-(methoxycarbonylmethyl)glutaric acid dimethylester as an oil yield (84.9 g, 94%).

NMR ¹H(CDCl₃), δ (12H, m, 4×CH₃), (2H, m, 2×CH₂) (1H, hextet, CH) 3.7 (1H, doublet, CH), (8H, 2 quartets, 4×CH₂O).

NMR ¹³C(CDCl₃), δ and 2×CH₃, CH, 2×CH2, CH; and 2×CH₂—O, 168.2 and 171.5 2×COO.

### Step 1 (c): Reduction and esterification of trimethyl ester to the triacetate

Under an atmosphere of nitrogen in a 3 necked 2 L round bottomed flask lithium aluminium hydride (20 g, 588 mmol) in tetrahydrofuran (400 ml) was treated cautiously with tri(methyloxycarbonylmethyl)methane (40 g, 212 mmol) in tetrahydrofuran (200 ml) over 1 h. A strongly exothermic reaction occurred, causing the solvent to reflux strongly. The reaction was heated on an oil bath at 90° C. at reflux for 3 days. The reaction was quenched by the cautious dropwise addition of acetic acid (100 ml) until the evolution of hydrogen ceased. The stirred reaction mixture was cautiously treated with acetic anhydride solution (500 ml) at such a rate as to cause gentle reflux. The flask was equipped for distillation and stirred and then heating at 90° C. (oil bath temperature) to distil out the tetrahydrofuran. A further portion of acetic anhydride (300 ml) was added, the reaction returned to reflux configuration and stirred and heated in an oil bath at 140° C. for 5 h. The reaction was allowed to cool and filtered. The aluminium oxide precipitate was washed with ethyl acetate and the combined filtrates concentrated on a rotary evaporator at a water bath temperature of 50° C. in vacuo (5 mmHg) to afford an oil. The oil was taken up in ethyl acetate (500 ml) and washed with saturated aqueous potassium carbonate solution. The ethyl acetate solution was separated, dried over sodium sulphate, and concentrated in vacuo to afford an oil. The oil was Kugelrohr distilled in high vacuum to give tris(2-acetoxyethyl)methane (45.313 g, 95.9% yield, 0.165 mol) as an oil. Bp. 220 at 0.1 mmHg.

NMR ¹H(CDCl₃), δ 1.66(7H, m, 3×CH₂, CH), 2.08(1H, s, 3×CH₃); 4.1 (6H, t 3×CH₂O).

NMR ¹³C(CDCl₃), δ 20.9, CH₃; 29.34, CH; 32.17, CH₂; 62.15, CH₂O; 171, CO.

### Step 1 (d): Removal of Acetate groups from the triacetate

Tris(2-acetoxyethyl)methane (45.3 g, 165 mM) in methanol (200 ml) and 880 ammonia (100 ml) was heated on an oil bath at 80° C. for 2 days. The reaction was treated with a further portion of 880 ammonia (50 ml) and heated at 80° C. in an oil bath for 24 h. A further portion of 880 ammonia (50 ml) was added and the reaction heated at 80° C. for 24 h. The reaction was then concentrated in vacuo to remove all solvents to give an oil. This was taken up into 880 ammonia (150 ml) and heated at 80° C. for 24 h. The reaction was then concentrated in vacuo to remove all solvents to give an oil. Kugelrohr distillation gave acetamide bp 170-180 0.2 mm. The bulbs containing the acetamide were washed clean and the distillation continued. Tris(2-hydroxyethyl)methane (22.53 g, 152 mmol, 92.1%) distilled at bp 220° C. 0.2 mm.

NMR ¹H(CDCl₃), δ 1.45(6H, q, 3×CH₂), 2.2(1H, quintet, CH); 3.7(6H, t 3×CH₂OH); 5.5(3H, brs, 3×OH).

NMR ¹³C(CDCl₃), δ 22.13, CH; 33.95, 3×CH₂; 57.8, 3×CH₂OH.

### Step 1 (e): Conversion of the triol to the tris(methanesulphonate)

To an stirred ice-cooled solution of tris(2-hydroxyethyl)methane (10 g, 0.0676 mol) in dichloromethane (50 ml) was slowly dripped a solution of methanesulphonyl chloride (40 g, 0.349 mol) in dichloromethane (50 ml) under nitrogen at such a rate that the temperature did not rise above 15° C. Pyridine (21.4 g, 0.27 mol, 4eq) dissolved in dichloromethane (50 ml) was then added drop-wise at such a rate that the temperature did not rise above 15° C., exothermic reaction. The reaction was left to stir at room temperature for 24 h and then treated with 5N hydrochloric acid solution (80 ml) and the layers separated. The aqueous layer was extracted with further dichloromethane (50 ml) and the organic extracts combined, dried over sodium sulphate, filtered and concentrated in vacuo to give tris(2-(methylsulphonyloxy)ethyl)methane contaminated with excess methanesulphonyl chloride. Theoretical yield was 25.8 g.

NMR ¹H(CDCl₃), δ 4.3 (6H, t, 2×CH₂), 3.0 (9H, s, 3×CH₃), 2 (1H, hextet, CH,), 1.85 (6H, q, 3×CH₂).

### Step 1(f): Preparation of 1,1,1-tris(2-azidoethyl)methane

A stirred solution of tris(2-(methylsulphonyloxy)-ethyl)methane [from step 1(e), contaminated with excess methylsulphonyl chloride] (25.8 g, 67 mmol, theoretical) in dry DMF (250 ml) under nitrogen was treated with sodium azide (30.7 g, 0.47 mol) portion-wise over 15 minutes. An exotherm was observed and the reaction was cooled on an ice bath. After 30 minutes, the reaction mixture was heated on an oil bath at 50° C. for 24 h. The reaction became brown in colour. The reaction was allowed to cool, treated with dilute potassium carbonate solution (200 ml) and extracted three times with 40/60 petrol ether/diethylether 10:1 (3×150 ml). The organic extracts were washed with water (2×150 ml), dried over sodium sulphate and filtered. Ethanol (200 ml) was added to the petrol/ether solution to keep the triazide in solution and the volume reduced in vacuo to no less than 200 ml. Ethanol (200 ml) was added and reconcentrated in vacuo to remove the last traces of petrol leaving no less than 200 ml of ethanolic solution.

CARE: DO NOT REMOVE ALL THE SOLVENT AS THE AZIDE IS POTENTIALLY EXPLOSIVE AND SHOULD BE KEPT IN DILUTE SOLUTION AT ALL TIMES.

NMR ¹H(CDCl₃), δ 3.35 (6H, t, 3×CH₂), 1.8 (1H, hextet, CH,), 1.6 (6H, q, 3×CH₂).

### Step 1(g): Preparation of 1,1,1-tris(2-aminoethyl)methane

Tris(2-azidoethyl)methane (15.06 g, 0.0676 mol), (assuming 100% yield from previous reaction) in ethanol (200 ml) was treated with 10% palladium on charcoal (2 g, 50% water) and hydrogenated for 12 h. The reaction vessel was evacuated every 2 hours to remove nitrogen evolved from the reaction and refilled with hydrogen. A sample was taken for NMR analysis to confirm complete conversion of the triazide to the triamine. Caution: unreduced azide could explode on distillation. The reaction was filtered through a celite pad to remove the catalyst and concentrated in vacuo to give tris(2-aminoethyl)methane as an oil. This was further purified by Kugelrohr distillation bp. 180-200° C. at 0.4 mm/Hg to give a colourless oil (8.1 g, 55.9 mmol, 82.7% overall yield from the triol).

NMR ¹H(CDCl₃), 2.72 (6H, t, 3×CH₂N), 1.41 (H, septet, CH), 1.39 (6H, q, 3×CH₂).

NMR ¹³C(CDCl₃), δ 39.8 (CH₂NH₂), 38.2 (CH₂.), 31.0 (CH).

### Step 1(h): Synthesis of bis[N-(1,1-dimethyl-2-N-hydroxyimine propyl)₂-aminoethyl]-(2-aminoethyl)methane (chelating agent 1)

To a solution of tris(2-aminoethyl)methane (4.047 g, 27.9 mmol) in dry ethanol (30 ml) was added potassium carbonate anhydrous (7.7 g, 55.8 mmol, 2eq) at room temperature with vigorous stirring under a nitrogen atmosphere. A solution of 3-chloro-3-methyl-2-nitrosobutane (7.56 g, 55.8 mol, 2eq) was dissolved in dry ethanol (100 ml) and 75 ml of this solution was dripped slowly into the reaction mixture. The reaction was followed by TLC on silica run in dichloromethane, methanol, concentrated (0.88sg) ammonia; 100/30/5 and the TLC plate developed by spraying with ninhydrin and heating. The mono, di and tri alkylated products were seen with RF's increasing in that order. Analytical HPLC was run using RPR reverse phase column in a gradient of 7.5-75% acetonitrile in 3% aqueous ammonia. The reaction was concentrated in vacuo to remove the ethanol and resuspended in water (110 ml). The aqueous slurry was extracted with ether (100 ml) to remove some of the trialkylated compound and lipophilic impurities leaving the mono and desired dialkylated product in the water layer. The aqueous solution was buffered with ammonium acetate (2eq, 4.3 g, 55.8 mmol) to ensure good chromatography. The aqueous solution was stored at 4° C. overnight before purifying by automated preparative HPLC.

Yield (2.2 g, 6.4 mM, 23%).

Mass spec; Positive ion 10 V cone voltage. Found: 344; calculated M+H=344.

NMR ¹H(CDCl₃), δ 1.24(6H, s, 2×CH₃), 1.3(6H, s, 2×CH₃), 1.25-1.75(7H, m, 3×CH₂,CH), (3H, s, 2×CH₂), 2.58 (4H, m, CH₂N), 2.88(2H, t CH₂N₂), 5.0 (6H, s, NH₂, 2×NH, 2×OH).

NMR ¹H ((CD₃)₂SO) δ 1.1 4×CH; 1.29, 3×CH₂; 2.1 (4H, t, 2×CH₂);

NMR ¹³C((CD₃)₂SO), δ 9.0 (4×CH₃), 25.8 (2×CH₃), 31.0 2×CH₂, 34.6 CH₂, 56.8 2×CH₂N, 160.3; C═N.

HPLC conditions: flow rate 8 ml/min using a 25 mm PRP column

A=3% ammonia solution (sp.gr=0.88)/water.

B=Acetonitrile

| Time | % B |
|---|---|
| 0 | 7.5 |
| 15 | 75.0 |
| 20 | 75.0 |
| 22 | 7.5 |
| 30 | 7.5 |

Load 3 ml of aqueous solution per run, and collect in a time window of 12.5-13.5 min.

### EXAMPLE 2

### Attachment of Chelating Agent 1 to 4-carboxy-N-(4-bromophenyl)-2-(4-chlorophenylsulfonylamido)benzamide to form Precursor 1

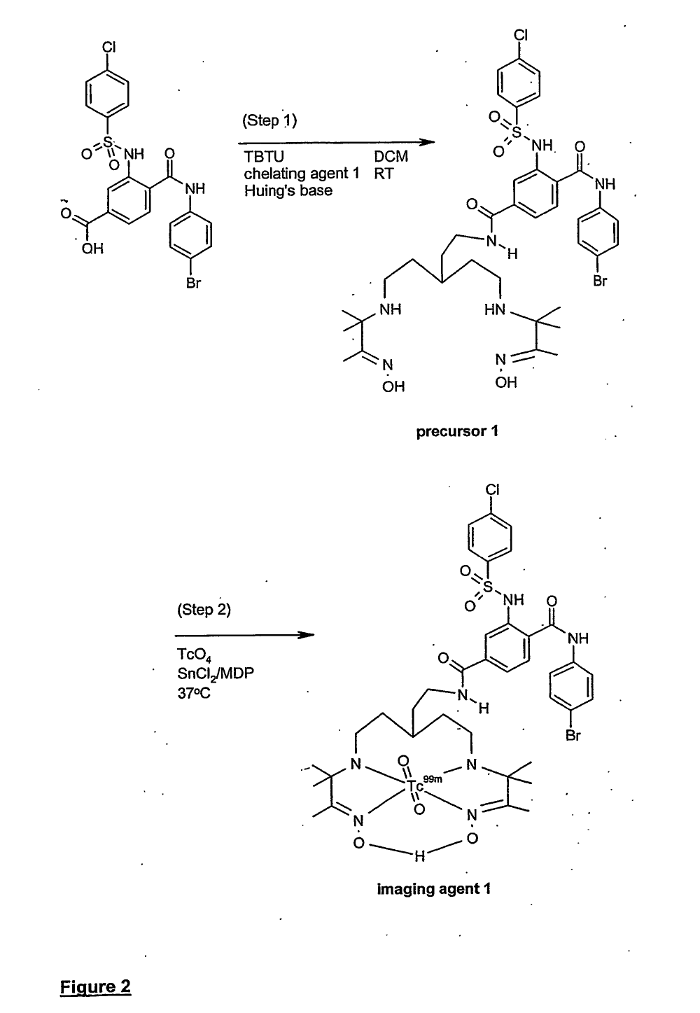

Chelating agent 1 was attached to 4-carboxy-N-(4-bromophenyl)-2-(4-chlorophenylsulfonylamido)benzamide by means of Step 1 of the reaction scheme depicted in FIG. 2.

To a solution of 4-carboxy-N-(4-bromophenyl)-2-(4-chlorophenylsulfonylamido) benzamide (1 mg) in dichloromethane (2 ml) at room temperature was added 4 equivalents of TBTU and 1.1 equivalent of chelating agent 1 of Formula (V), and 3 equivalents of N,N-diisopropylethylamine (DIEA) under a nitrogen atmosphere for 24 hours. The crude mixture was purified by HPLC.

Mass Spectrometry analysis: ES [M+H] m/z 836.

### EXAMPLE 3

### Attachment of Chelating Agent 1 to 4-bromo-N-(4-bromophenyl)-2-(4-carboxyphenylsulfonylamido)benzamide to Form Precursor 2

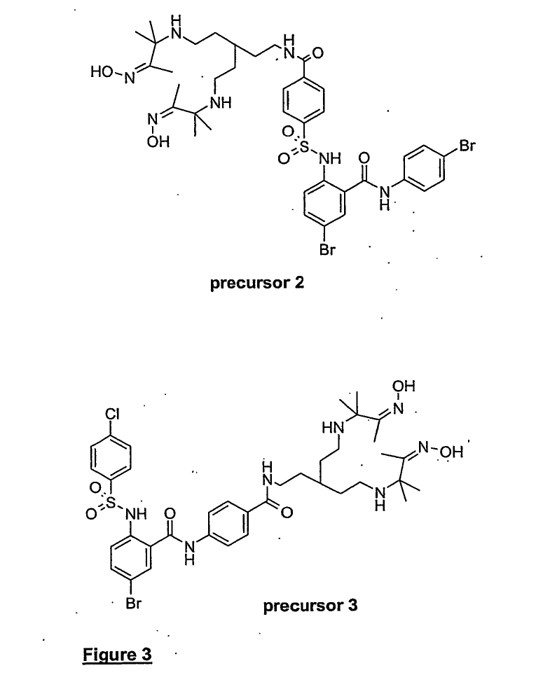

The method described in Example 2 was used to attach chelating agent 1 to 4-bromo-N-(4-bromophenyl)-2-(4-carboxyphenylsulfonylamido)benzamide to form precursor 2, which is illustrated in FIG. 3.

### EXAMPLE 4

### Attachement of Chelating Agent 1 to 4-bromo-N-(4-carboxyphenyl)-2-(4-chlorophenylsulfonylamido)benzamide to Form Precursor 3

The method described in Example 2 was used to attach chelating agent 1 to 4-bromo-N-(4-carboxyphenyl)-2-(4-chlorophenylsulfonylamido)benzamide to form precursor 3, which is illustrated in FIG. 3.

### EXAMPLE 5

### ^{99m}Tc Labelling of Precursor 1 to Form Imaging Agent 1

Imaging agent 1 was prepared by labelling precursor 1 with ^{99m}Tc according to Step 2 of the reaction scheme depicted in FIG. 2.

A SnCl₂/MDP solution was prepared by dissolving 10 mg SnCl₂ and 90 mg MDP in 100 ml of nitrogen-purged saline. To 50 μl 1 mg/ml in methanol of precursor 1, was added; (1) 0.7 ml methanol, (2) 0.5 ml 0.1M sodium carbonate buffer, (3) 0.5 ml 500 MBq/ml TcO₄, and (4) 100 μl of the SnCl₂/MDP solution. This reaction mixture was heated at 37° C. for 30 min to form imaging agent 1. The activity of solution was 185 MBq.

An ITLC (instant thin layer chromatography) using silica gel plates and a mobile phase of MeOH/(NH₄OAc 0.1 M) 1:1 showed 3% RHT (reduced hydrolysed technetium) at the origin. HPLC analysis showed 88% of imaging agent 1 to give an RCP of 85%. The retention time of imaging agent 1 was 16.6 min.

HPLC analyses was carried out using an Xterra RP18, 3.5 μm, 4.6×150 mm column using an aqueous mobile phase (solvent A) of 0.06% NH₄OH and organic mobile phase (solvent B) of acetonitrile and a flow rate of 1 ml/min. Typical gradients used were as follows: 0-5 min (10-30% B), 5-20 min (30% B), 20-21 min (30-100% B), 21-25 min (100% B) and 25-27 min (100-10% B).

### EXAMPLE 6

### Synthesis of Precursor 4

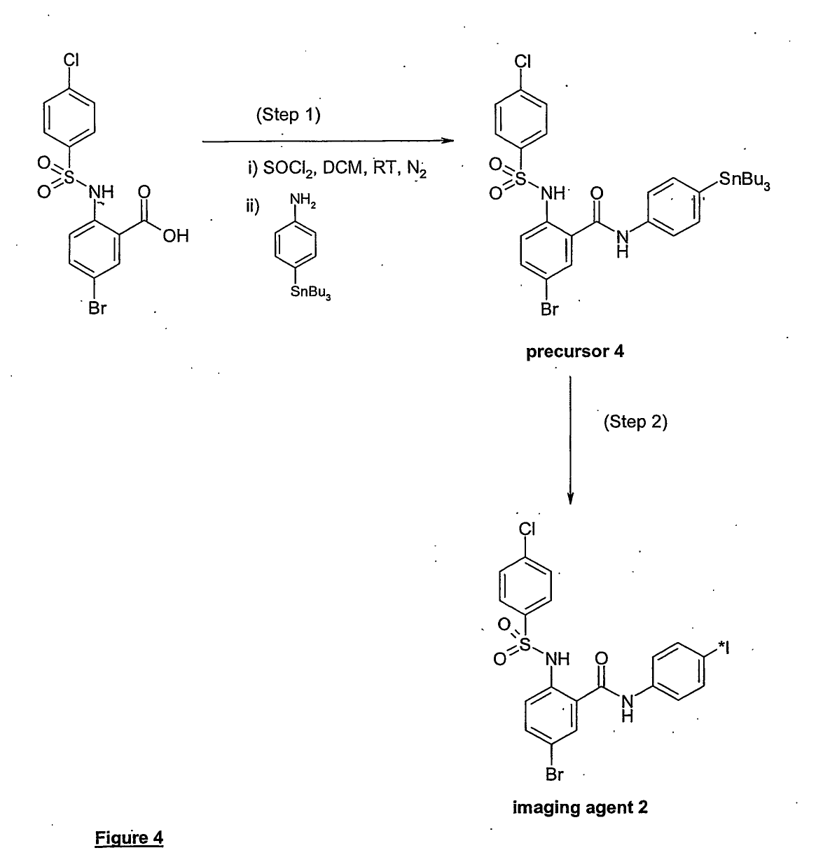

Step 1 of the reaction scheme depicted in FIG. 4 is used to prepare precursor 4. 4-n-tributyltin aniline is coupled to 5-bromo-2-(4-chlorophenylsulfonamido)benzoic acid in DCM in the presence of 1.5 equivalents of triethylamine to give precursor 4.

### EXAMPLE 7

### Radioiodination of Precursor 4 to Form Imaging Agent 2

Radioiodination of precursor 4 to form imaging agent 2 is carried out according to Step 2 of the scheme depicted in FIG. 4.

10 μM of precursor 4 is reacted with 0.05 mL NaI solution [approx. 0.167 μM total radioiodine (I*)] in the presence of 0.4 mL DMF, 0.1 mL ammonium acetate buffer (pH 4, 0.2 M) and 0.05 mL Chloramine-T solution (0.22 μM). 0.5 mL H₂O is added after 5 minutes. The crude mixture is subsequently separated by HPLC to yield pure imaging agent 2.

### EXAMPLE 8

### Synthesis of Precursor 5

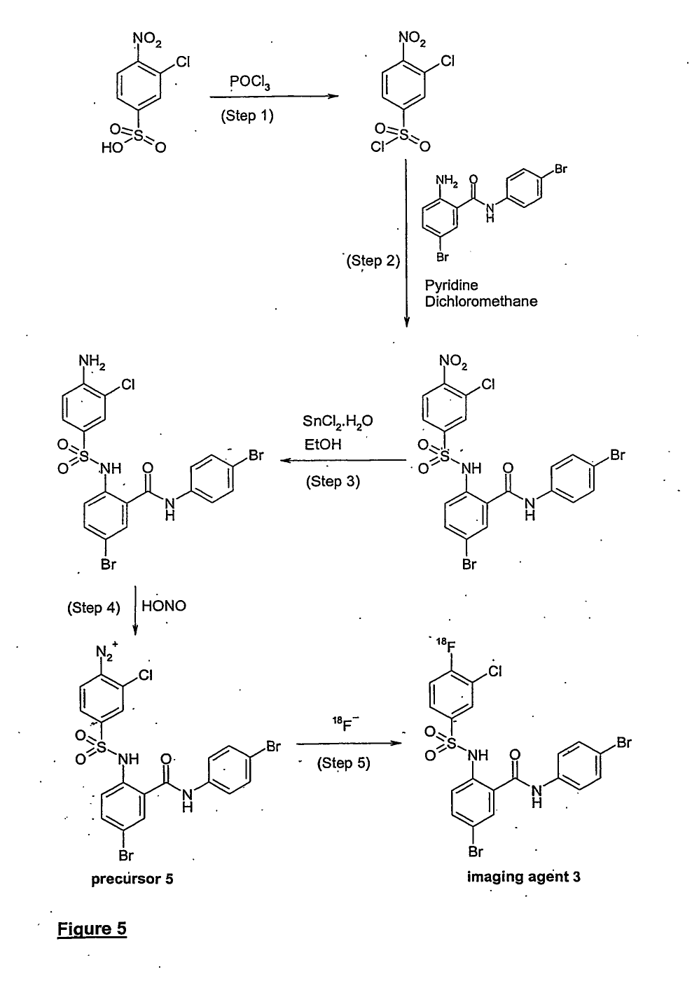

In Step 1 of the reaction scheme illustrated in FIG. 5, 3-chloro-4-nitrobenzenesulfonic acid is reacted with POCl₃ to form 3-chloro-4-nitrobenzenesulfonyl chloride, which is reacted with N-(4-bromophenyl)-2-amino-5-bromobenzamide to form 4-bromo-N-(4-bromophenyl)-2-(3-chloro-4-nitro-phenyl sulphonamido)benzamide (Step 2). The nitro group is then reduced with SnCl₂.2H₂O to yield the amine (Step 3). The amine is then converted into the diazonium compound by treatment with nitrous acid (HONO) in Step 4 to form precursor 5.

### EXAMPLE 9

### Synthesis of Imaging Agent 3

In Step 5 of the scheme illustrated in FIG. 4 ¹⁸F⁻ is reacted with the diazonium compound to give imaging agent 3.

### EXAMPLE 10

### Preparation of Non-Radioactive Imaging Agent 2

A solution of N-(4-iodophenyl)-2-amino-5-bromobenzamide (3 mmol), 4-chlorobenzenesulfonyl chloride (3 mmol) and pyridine (12 mmol) in dichloromethane (20 ml) was stirred for 18 hours under a nitrogen atmosphere. The solvent was removed under vacuum and the residue dissolved in methanol. Purification was done by HPLC (C 18, 20-95% acetonitrile −0.1% aqueous trifluoroacetic acid).

After HPLC purification the product gave the required ¹H NMR and MS analysis.

¹H NMR (CD₃OD) δ 7.32 (2H, d, J=4.5 Hz), 7.38 (2H, d, J=4.5 Hz), 7.52 (1H, d, J=4.5 Hz), 7.59 (2H, d, J=4.5 Hz), 7.65-7.70 (3H, m), 7.82 (1H, d, J=1 Hz)

MS (ES-ve) m/z=591.

### EXAMPLE 11

### Preparation of Non-Radioactive Imaging Agent 3

A solution of N-(4-bromophenyl)-2-amino-5-bromobenzamide (3 mmol), 3-chloro-4-fluorobenzenesulfonyl chloride (3 mmol) and pyridine (12 mmol) in dichloromethane (20 ml) was stirred for 18 hours under a nitrogen atmosphere. The solvent was removed under vacuum and the residue dissolved in methanol. Purification was done by HPLC (C 18, 20-95% acetonitrile-0.1% aqueous trifluoroacetic acid).

After HPLC purification the product gave the required ¹H NMR analysis.

¹H NMR (CD₃OD) δ 7.20 (1H, t, J=9 Hz), 7.46-7.59 (6H, m), 7.68 (1H, dd, J=3 and 6 Hz), 7.80 (1H, dd, J=2.4 and 4.8 Hz), 7.84 (1H, d, J=1.1 Hz)

### EXAMPLE 12

### Scavenger Receptor Binding Assay

An assay was developed based on that described in Lysko et al. [1999 J. Pharmacol Exp Ther 289 (3); 1277-1285]. The cells used in the assay were either mouse J774.1 or human THP-1. J774.1 cells were seeded at 1×10⁵ cells/well/ml 24 hours prior to assay in Dulbecco's minimum essential medium containing penicillin/streptomycin, 2 mM glutamine and 10% foetal bovine serum. THP-1 cells were seeded at 1×10⁵ cells/well/ml in RPMI-1640 medium containing penicillin/streptomycin, 2 mM glutamine and 10% foetal bovine serum with 400 ng/ml phorbol myristate acetate 4-6 days prior to assay. For the assay, the medium was decanted from the plates and they were washed with 1 ml/well ice cold phosphate-buffered saline containing 2 mg/ml BSA. Into the wells, add the following reagents (all in μl):

| | NSB well | Bo well | Assay well |
|---|---|---|---|
| Assay buffer | 100 | 150 | 100 |
| Competing | — | — | 50 |
| compound | | | |
| acLDL (for NSB*) | 50 | — | — |
| [¹²⁵I]acLDL | 50 | 50 | 50 |

*NSB = non-specific binding

The assay buffer was Dulbecco's minimal essential medium containing penicillin/streptomycin, 2 mM glutamine and 2 mg/ml bovine serum albumin. AcLDL was obtained from Biogenesis (Cat no. 5685-3404). [¹²⁵I]acLDL (Biogenesis, Cat. no. 5685-3502) used at 150,000 cpm per well in assay (approx. 1.5 μg/ml).

The plates were incubated for 3 hours at 37° C. after which time the reagents were removed and the plates were washed with pre-chilled wash buffer (2: 0.15M NaCl, 50 mM Tris-HCl, pH7.4). The plates were then incubated with pre-chilled wash buffer for 10 minutes on ice, and this step was then repeated. A further rapid wash was carried out with a different wash buffer (0.15M NaCl, 50 mM Tris-HCl, pH7.4) before adding 500 μl NaOH for 30 minutes at room temperature. The well contents were transferred to Sarstedt tubes for radioactivity counting on a Wallac 1480 Wizard automatic gamma counter.

In order to assess cell coverage in the wells and show that cells were not lost during assay, 300 μl of a 2% crystal violet stain in 95% methanol was added to the wells and incubated for 30 minutes at room temperature.

The IC50 for non-radioactive imaging agent 2 was 25.2 μM, and chemically identical radioiodinated versions of the compound should produce similar values. The IC50 for non-radioactive imaging agent 3 was found to be 25.9 μM, and the chemically identical ¹⁸F labelled version of the compound should produce a similar value.

### EXAMPLE 13

### Spiked Saturation Binding Assay

Experiments were carried out on THP-1 cells. THP-1 cells (human monocyte ECACC) are suspension cells and were cultured in RPMI-1640 medium (Sigma, Cat no. R0883) containing penicillin/streptomycin (Sigma, Cat no. P4458), 2 mM glutamine (Sigma, Cat no. G7513) and 10% foetal bovine serum (FBS; Sigma, Cat no. F-7524). Cells were routinely passaged at 2×10⁵ cells/ml in 162 cm² flasks. For assays with ¹²⁵I-acLDL, cells were seeded at 1×10⁵ cells/well/ml in 24 well plates 4-6 days prior to assay and 400 ng/ml phorbol myristate acetate (PMA) added to differentiate THP-1 cells. Cells became adherent and expressed MSRA. For assays with ¹²⁵I imaging agent 2, THP-1 cells were seeded at 1.5×10⁵ cells/well/ml in 24 well plates 4-6 days prior to assay with 400 ng/ml PMA.

¹²⁵I imaging agent 2 was diluted to a concentration of 400,000 cpm/50 μl in assay buffer. Non-radioactive imaging agent 2 was prepared at 1 mg/ml stock in 12.5% DMSO in assay buffer according to the method described in Example 10. For non-specific binding (NSB) measurement, a 1:2 dilution of this stock was prepared.

For spiked samples, two series of non-radioactive imaging agent 2 dilutions were also setup. For series A, a starting concentration of 0.125 mg/ml (or 0.031 mg/ml per well) was prepared, and 15 further doubling dilutions were made. For series B, a starting concentration of 0.188 mg/ml (or 0.047 mg/ml per well) was prepared, and 15 further doubling dilutions were made.

Media was decanted from plates and wells washed in ice cold PBS at 1 ml/well. PBS was removed by decanting and any residual liquid removed with a pipette tip.

Assay reagents were then added as below:
100% assay buffer (except in wells without non-radioactive imaging agent 2 where 150 μl assay buffer was added)
50 μl non-radioactive imaging agent 2 (appropriate dilution)
50 μl ¹²⁵I imaging agent 2

Plates were incubated for 3 hr, 37° C. Assay reagents were removed with a pipette tip and wells washed twice with 1 ml pre-chilled wash buffer 1. Plates were then incubated with 1 ml/well pre-chilled wash buffer 1 for 10 min on ice. Wash buffer was decanted and a further 10 min incubation on ice with wash buffer 1 carried out. A rapid wash with 1 ml/well pre-chilled wash buffer 2 on ice was then carried out. Trypan blue (200 μl of a 1:5 dilution in PBS) was then added to each well to check for toxicity. 500 μl 0.1M NaOH was then added and plates left for 30 min, R.T. Well contents were then transferred to Sarstedt tubes and counted on a Wallac Wizard.

### EXAMPLE 14

### Cell Competition Assay with ¹²⁵I-Imaging Agent 2

Experiments were carried out in THP-1 and CHO-SRA cells to assess competition for binding of ¹²⁵I imaging agent 2 by acLDL, oxLDL and non-radioactive imaging agent 2.

THP-1 cells were cultured as described in Example 13 above.

CHO-SRA cells are adherent hamster ovary cells expressing human SR-A and were cultured in Hams F-12 medium containing penicillin/streptomycin, 2 mM glutamine, HEPES buffer (7 ml per 500 ml media), 3% filter-sterilised lipoprotein-deficient serum (LPDS; Sigma cat. no. S5394), 40 μM compactin/mevastatin (Sigma cat. no. M2537), 250 μM mevalonic acid lactone (Sigma cat. no. M4667), and 3 μg/ml acetylated LDL (AcLDL; Biogenesis, Cat no. 5685-3404). For assays with ¹²⁵I-acLDL, cells were seeded at 1×10⁵ cells/well/ml in 24 well plates 24 hours prior to assay. For assays with ¹²⁵I imaging agent 2, CHO-SRA cells were seeded at 1.5×10⁵ cells/well/ml in 24 well plates 24 hours prior to assay.

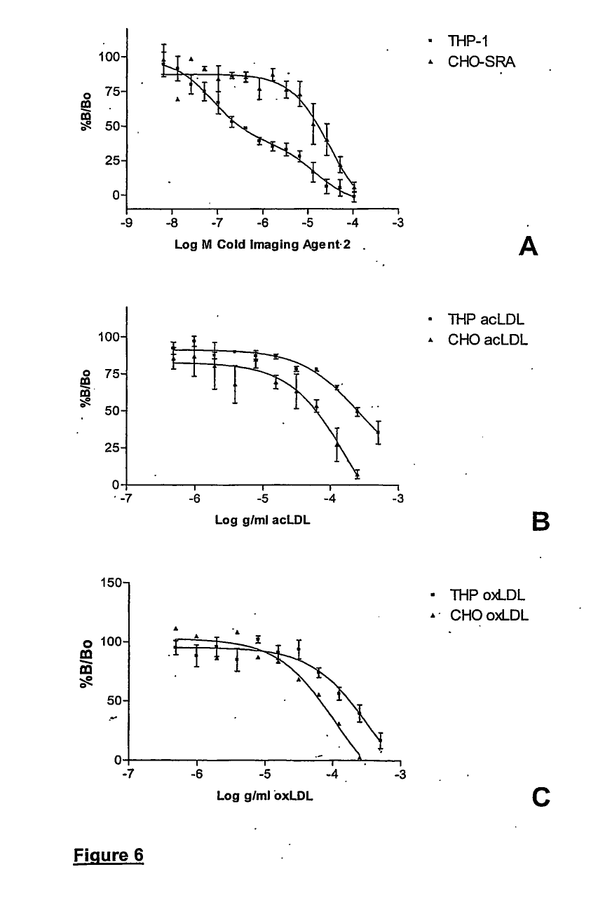

In THP-1 cells, non-radioactive imaging agent 2 competed for binding of ¹²⁵I imaging agent 2 with two site kinetics (FIG. 6A). Mean IC₅₀ values of 79±20 nM (n=3) and 14.5±5.8 μM (n=3) were obtained. However in CHO-SRA cells one site competition was observed with a mean IC₅₀ value of 30±12 μM (n=3).

AcLDL (FIG. 6B) and oxLDL (FIG. 6C) both displayed one site competition for binding of ¹²⁵I imaging agent 2 in THP-1 and CHO-SRA cells. Mean IC₅₀ values for acLDL were ˜281 μg/ml (n=3) in THP-1 cells and 192 μg/ml (n=2) in CHO-SRA cells. Mean IC₅₀ values for oxLDL were ˜369 μg/ml (n=2) in THP-1 cells and 112 μg/ml (n=1) in CHO-SRA cells.

### EXAMPLE 15

### In Vivo Assessment of Imaging Agent 2

J774 cells are mouse macrophage cells that express MSRA and MSRB (macrophage scavenger receptor B). A J774 tumour model was utilised for screening of MRSA targeted vectors. These cells have been utilised to generate in vivo tumours in BALB/C mice (Ralph et al., 1975 J. Immunol. 114(2) pp898-905). J774 tumours were inoculated subcutaneously in BALB/C male mice with biodistribution studies performed 24-28 days post inoculation.

0.1 ml of ¹²⁵I imaging agent 2 was injected intravenously as bolus via the tail vein of tumoured mice, with animals euthanised at 5, 30, 60, 120 and 240 minutes p.i.

What is claimed is:

1) An imaging agent which comprises a synthetic MSRA antagonist labelled with an imaging moiety, wherein the synthetic MSRA antagonist is a sulphonamidobenzamide compound, and wherein the imaging moiety can be detected externally in a non-invasive manner following administration of said labelled synthetic MSRA antagonist to the mammalian body in vivo.

2) The imaging agent of claim 1 wherein the sulphonamidobenzamide compound is of Formula (II): 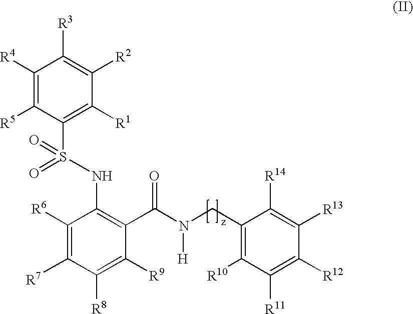
wherein;
z is 0, 1 or 2;
R¹-R¹⁴ are independently R groups, where R is;
hydrogen, hydroxy, carboxy, C₁₋₆ alkyl, nitro, cyano, amino, halogen, C₆₋₁₄ aryl, C₂₋₇ alkenyl, C₂₋₇ alkynyl, C₁₋₆ acyl, C₇₋₁₅ aroyl, C₂₋₇ carboalkoxy, C₂₋₁₅ carbamoyl, C₂₋₁₅ carbamyl, C₁₋₆ alkysulphinyl, C₆₋₁₄ arylsulphinyl, C₆₋₁₂ arylalkylsulphinyl, C₁₋₆ alkylsulphonyl, C₆₋₁₄ arylsulphonyl, C₆₋₁₂ arylalkylsulphonyl, sulphamyl, C₆₋₁₄ arylsulphonamido or C₁₋₆ alkylsulphonamido.

3) The imaging agent of claim 2 wherein each R¹ to R¹⁴ is chosen from:
an imaging moiety, hydrogen, C₁₋₆ alkyl, hydroxy, carboxy, amino or halogen.

4) The imaging agent of claim 2 wherein one of R², R³, R⁷, R⁸ and R¹² in Formula (II) is an imaging moiety, and the remaining R², R³, R⁷, R⁸ and R¹² groups are independently selected from hydrogen, C₁₋₆ alkyl, carboxy, or a halogen selected from chlorine, bromine, fluorine or iodine.

5) The imaging agent of claim 2 wherein R³, R⁸ and R¹² are each independently a halogen selected from chlorine, bromine, fluorine or iodine.

6) The imaging agent of claim 1, wherein said imaging moiety is selected from:
(i) a radioactive metal ion;
(ii) a paramagnetic metal ion;
(iii) a gamma-emitting radioactive halogen;
(iv) a positron-emitting radioactive non-metal;
(v) a hyperpolarised NMR-active nucleus;
(vi) a reporter suitable for in vivo optical imaging;
(vii) a □-emitter suitable for intravascular detection.

7) The imaging agent of claim 6, wherein the radioactive metal ion is a gamma emitter or a positron emitter.

8) The imaging agent of claim 7, wherein the radioactive metal ion is selected from ^{99m}Tc, ^{94m}Tc, ¹¹¹In, ^{113m}In, ⁶⁴Cu, ⁶⁷Cu, ⁶⁷Ga, ⁶⁸Ga, ⁴⁸V, ⁵²Fe and ⁵⁵Co.

9) The imaging agent of claim 6, wherein the paramagnetic metal ion is selected from paramagnetic ions of Gd, Mn and Fe.

10) The imaging agent of claim 7, wherein the paramagnetic metal ion is Gd(III).

11) The imaging agent of claim 6, wherein the gamma-emitting radioactive halogen is a radioactive isotope of iodine.

12) The imaging agent of claim 11, wherein the radioactive isotope of iodine is chosen from ¹²³I or ¹³¹I.

13) The imaging agent of claim 6, wherein the positron-emitting radioactive non-metal is selected from ¹¹C, ¹³N, ¹⁵O, ¹⁷F, ¹⁸F, ¹²⁴I, ⁷⁵Br and ⁷⁶Br.

14) The imaging agent of claim 13, wherein the positron-emitting radioactive non-metal is ¹⁸F.

15) The imaging agent of claim 6 wherein the hyperpolarised NMR-active nucleus is selected from ¹³C, ¹⁵N, ¹⁹F, ²⁹Si and ³¹P.

16) The imaging agent of claim 15 wherein the hyperpolarized NMR-active nucleus is ¹³C.

17) The imaging agent of claim 6, wherein the imaging moiety is a radioactive or a paramagnetic metal ion and the metal ion is attached to the MSRA antagonist as part of a metal complex to form a conjugate of Formula (III):
[{MSRA antagonist}-(L)ₓ]_{y}-[metal complex] (III)
wherein:
-(L)ₓ- is a linker group wherein each L is independently —CZ₂-, —CZ=CZ-, C≡C—, —CZ₂CO₂—, —CO₂CZ₂-, —NZCO—, —CONZ-, —NZ(C═O)NZ-, —NZ(C═S)NZ-, —SO₂NZ-, —NZSO₂—, —CZ₂OCZ₂-, —CZ₂SCZ₂-, —CZ₂NZCZ₂-, a C₄₋₈ cycloheteroalkylene group, a C₄₋₈ cycloalkylene group, a C₅₋₁₂ arylene group, a C₃-12 heteroarylene group, an amino acid or a monodisperse polyethyleneglycol (PEG) building block;
Z is independently chosen from H, C₁₋₄ alkyl, C₂₋₄ alkenyl, C₂₋₄ alkynyl, C₁₋₄ alkoxyalkyl or C₁₋₄ hydroxyalkyl;
x is an integer of value 0 to 10; and
y is 1, 2 or 3.

18) The imaging agent of claim 17 wherein the metal complex is a coordination complex of the radioactive metal ion or the paramagnetic metal ion with one or more ligands.

19) The imaging agent of claim 18 wherein said one or more ligands are chelating agents selected from diaminedioximes, N₃S ligands, N₂S₂ ligands, N₄ ligands and N₂O₂ ligands.

20) An imaging agent precursor of Formula (IIIa):
[{MSRA antagonist}-(L)ₓ]_{y}-[ligand] (IIIa)
wherein:
(L)ₓ is a linker group wherein L is as defined in claim 17;
x is an integer of value 0 to 10; and
y is 1, 2 or 3.

21) A pharmaceutical composition comprising the imaging agent of claim 1, together with a biocompatible carrier, in a form suitable for mammalian administration.

22) The pharmaceutical composition of claim 21 for use in the diagnostic imaging of cardiovascular disease.

23) The pharmaceutical composition of claim 21 for use in the diagnostic imaging of atherosclerotic plaques, coronary artery disease, thrombosis, transient ischaemia or renal disease.

24) The pharmaceutical composition of claim 23 for use in the diagnostic imaging of atherosclerotic plaques.

25) The pharmaceutical composition of claim 24 for use in the diagnostic imaging of unstable atherosclerotic plaques.

26) A kit for the preparation of the pharmaceutical composition of claim 21, comprising a precursor of the imaging agent of claim 1.

27) The kit of claim 26 wherein said precursor is of Formula (IIIa) of claim 20.

28) The kit of claim 27 wherein the preparation of said pharmaceutical composition comprises reaction of a radioactive metal ion or a paramagnetic metal ion with the precursor of Formula (IIIa).

29) The kit of claim 28 wherein the radioactive metal ion is selected from ^{99m}Tc, ¹¹¹In, ⁶⁴Cu, ⁶⁷Cu, ⁶⁷Ga and ⁶⁸Ga.

30) The kit of claim 28 wherein the radioactive metal ion is ^{99m}Tc.

31) The kit of claim 28 wherein the paramagnetic metal ion is selected from Gd, Mn and Fe.

32) The kit of claim 31 wherein the paramagnetic metal ion is Gd(III).

33) Use of the imaging agent of claim 1 for the diagnostic imaging of cardiovascular disease.

34) The use of claim 33 wherein the cardiovascular disease is atherosclerosis.
